(19) United States
(12) Patent Application Publication
(10) Pub. No.: US 2015/0259738 A1
(43) Pub. Date: Sep. 17, 2015

(54) CALIBRATORS FOR USE IN HIGH-RESOLUTION MELT ANALYSIS AND RELATED METHODS

(71) Applicant: University of Utah Research Foundation, Salt Lake City, UT (US)

(72) Inventors: Roberto Nussenzveig, South Jordan, UT (US); Archana M. Agarwal, Holladay, UT (US); Ha T. Pham, West Jordan, UT (US); Steven F. Dobrowolski, Park City, UT (US)

(21) Appl. No.: 14/249,099

(22) Filed: Apr. 9, 2014

Related U.S. Application Data
(60) Provisional application No. 61/853,626, filed on Apr. 9, 2013.

(51) Int. Cl. C12Q 1/68

(52) U.S. Cl. CPC C12Q 1/6876; C12Q 2600/166; C12Q 2600/156

(57) ABSTRACT

Calibrators for use in high-resolution melt analysis and related methods are disclosed and described. In one embodiment a polynucleotide thermal melt calibrator having a stem-loop configuration may include: a first stem region having a first nucleotide sequence; a second stem region having a second nucleotide sequence; a loop region joining the first stem region and the second stem region; and a hybridization indicator associated with at least one of the first stem region, the second stem region, or the loop region.

### PRIORITY DATA

This application claims the benefit of U.S. Provisional Patent Application Ser. No. 61/853,626, filed Apr. 9, 2013, which is incorporated herein by reference.

### BACKGROUND OF THE DISCLOSURE

Significant advances in instrumentation and methods have made high resolution melting (HRM) a powerful, cost-effective, high-throughput tool for genotyping. Single closed-tube genotyping of small amplicons by HRM using DNA intercalating fluorescent dyes is a rapid low-cost alternative to methods such as pyrosequencing or use of labeled oligonucleotide probes. Yet variables such as DNA extraction methods and concentrations lead to within-run sample melt discrepancies. Moreover, homozygous base-pair neutral single nucleotide polymorphisms (SNP), in particular those in classes 3 and 4, can be difficult to genotype accurately due to the small differences in melt temperature (Tm).

### BRIEF DESCRIPTION OF THE FIGURES

FIGS. 1A-D show HRM data according to one aspect of the present disclosure.

FIGS. 2A-F show HRM data according to another aspect of the present disclosure.

FIGS. 3A-C show stem loop sequences and HRM data according to another aspect of the present disclosure.

FIGS. 4A-D show HRM data according to another aspect of the present disclosure.

FIGS. 5A-B show HRM data according to another aspect of the present disclosure.

### DESCRIPTION OF EMBODIMENTS

It has been discovered that use of two complementary oligonucleotides as melt calibrators has proven useful for discrimination of temperature shift neutral polymorphisms (A>T, T>A, G>C, and C>G), including for DNA samples from different sources (e.g. fresh tissue and FFPE), purified by different procedures, or at different concentrations. This is of great value for non-base pair neutral changes in order to tighten clustering profiles for estimation of allele copy-number. At present, melt calibrators are mostly used for the later purpose. Melt calibrators may also be used for estimation of mutant allele burden (e.g. cancer or mitochondrial heteroplasmy. Melt calibrators based on formation of blunt-end stem loop structures and single oligonucleotides have been developed, including hairpin melt calibrators of both low and high Tm. Current technology is based on the use of 3′end blocked PAGE purified complementary oligonucleotides (total of 4) for low and high Tm calibration. The cost of each blocked and purified oligonucleotide is between $200-$300 (total cost for both calibrators ˜$1000), a minimum scale of 100 nmoles, and completed product yield of less than 5% (5 nmoles) is obtained.

As used in this specification, “comprises,” “comprising,” “containing” and “having” and the like can have the meaning ascribed to them in U.S. Patent law and can mean “includes,” “including,” and the like, and are generally interpreted to be open ended terms. The term “consisting of” or “consists of” is a closed term, and includes only the components, structures, steps, processes, compositions, systems, or the like specifically listed, and that which is in accordance with U.S. Patent law. “Consisting essentially of” or “consists essentially” are generally closed terms, limiting the components, structures, steps, processes, compositions, systems, or the like, when applied to methods, compositions, or systems specifically listed, as well as other elements that do not substantially alter or effect the basic and novel characteristics of the item to which the “consisting essentially of” language refers. In further detail, “consisting essentially of” or “consists essentially” or the like, when applied to components, structures, steps, processes, compositions, systems, or the like encompassed by the present disclosure have the meaning ascribed in U.S. Patent law. When using an open ended term, like “comprising” or “including,” it is understood that direct support should be afforded also to “consisting essentially of” language as well as “consisting of” language as if stated explicitly and vice versa.

The terms “first,” “second,” “third,” “fourth,” and the like in the description and in the claims, if any, are used for distinguishing between similar elements and not necessarily for describing a particular sequential or chronological order. It is to be understood that the terms so used are interchangeable under appropriate circumstances such that the embodiments described herein are, for example, capable of operation in sequences other than those illustrated or otherwise described herein. Similarly, if a method is described herein as comprising a series of steps, the order of such steps as presented herein is not necessarily the only order in which such steps may be performed, and certain of the stated steps may possibly be omitted and/or certain other steps not described herein may possibly be added to the method. Furthermore, the terms “comprise,” “include,” “have,” and any variations thereof, are intended to cover a non-exclusive inclusion, such that a process, method, article, or apparatus that comprises a list of elements is not necessarily limited to those elements, but may include other elements not expressly listed or inherent to such process, method, article, or apparatus.

As used herein, the term “about” is used to provide flexibility to a numerical range endpoint by providing that a given value may be “a little above” or “a little below” the endpoint.

As used herein, “substantial” and “substantially” when used in reference to a quantity or amount of a material, or a specific characteristic thereof, refers to an amount that is sufficient to provide an effect that the material or characteristic was intended to provide. The exact degree of deviation allowable may in some cases depend on the specific context. Similarly, “substantially free of” or the like refers to the lack of an identified element or agent in a composition. Particularly, elements that are identified as being “substantially free of” are either completely absent from the composition, or are included only in amounts which are small enough so as to have no measurable effect on the composition.

Reference throughout this specification to “an example” means that a particular feature, structure, or characteristic described in connection with the example is included in at least one embodiment. Thus, appearances of the phrases “in an example” in various places throughout this specification are not necessarily all referring to the same embodiment.

As used herein, a plurality of items, structural elements, compositional elements, functions, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, levels and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges or decimal units encompassed within that range as if each numerical value and sub-range is explicitly recited. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc., as well as 1, 2, 3, 4, and 5, individually. This same principle applies to ranges reciting only one numerical value as a minimum or a maximum. Furthermore, such an interpretation should apply regardless of the breadth of the range or the characteristics being described.

With the above-recited information in mind, the inventors have developed melt calibrators that are simple, accurate, and inexpensive. Embodiments of the present disclosure can be synthesized at a scale as low as 25 nmoles, require no modification or purification, and can cost as little as ˜$10 (total for both calibrators ˜$20). Moreover, annealing and melt of complementary oligonucleotides follows the physical principles of Brownian motion and random collision making such events dependent on oligonucleotide concentration. In contrast, such limitations do not affect the present calibrator design embodiments since the complementary sequences within the stem structure are always close by and their annealing thus follows a 0th order reaction.

In addition, a hypothesis that stem-loop formation would be favored over base-pairing of the complementary segments of two oligonucleotides has been confirmed experimentally. The presence of 4 oligonucleotides (low and high) in addition to the gene specific primers during PCR and HRM increases the complexity of the reaction and makes it more susceptible to failure. Embodiments of the present disclosure may be platform independent and can be used for both research purposes as well as clinical diagnostic testing. The low cost for synthesis and concentration independence facilitates robust signal detection across platforms and makes the invention embodiments very useful in the clinical setting. In addition, samples from distinct sources (e.g. fresh tissue or FFPE) can be screened simultaneously and the results calibrated/normalized. After significant testing several oligonucleotide stem-loop designs calibrators for both low and high melt analysis have been produced. Additional oligonucleotide stem-loop calibrators at distinct melting Tm's can be prepared.

In one aspect of the present disclosure, for example, a polynucleotide thermal melt calibrator having a stem-loop configuration is provided. A variety of sequence configurations are contemplated, and calibrator sequences can be designed to achieve a variety of melt profiles. In general, sequences that are A-T rich have a lower Tm as compared to sequences that are G-C rich. In addition, the loop structure coupling the base pairing stem sequences together can affect melt temperature and the shape of the melting profile due to, among other things, the physical linking of the stem sequences.

As such, in one aspect a thermal melt calibrator can include a first stem region having a first nucleotide sequence and a second stem region having a second nucleotide sequence, with a loop region joining the first stem region and the second stem region. In one aspect, the second nucleotide sequence is sufficiently complimentary to the first nucleotide sequence to at least substantially hybridize and at least substantially denature across a temperature range of about 10° C. or less. Thus, in macroscopic terms, the stem region transitions from an unmelted state to a melted state over a 10° C. range or less. In another aspect, the second nucleotide sequence is sufficiently complimentary to the first nucleotide sequence to at least substantially hybridize and at least substantially denature across a temperature range of about 5° C. or less. In yet another aspect, the second nucleotide sequence is sufficiently complimentary to the first nucleotide sequence to at least substantially hybridize and at least substantially denature across a temperature range of about 2° C. or less.

As has been described, the calibrators include a loop region linking the first and second stem regions together. The loop region has a loop sequence and a loop length to allow hybridization of the first stem region and the second stem region. The design of various loop sequences and loop lengths would readily be understood by those of ordinary skill in the art once in possession of the present disclosure. In addition, in some aspects the melt calibrator can include a hybridization indicator associated with at least one of the first stem region, the second stem region, or the loop region. The hybridization indicator thus allows the melting profile to be measured via a number of indicator techniques.

It is noted that the following sequences are merely exemplary, and that the present scope includes variations in such sequences that allow the stem loop calibrators to achieve a given melt profile. In one specific aspect, the first nucleotide sequence can be one of SEQ ID 001, SEQ ID 002, SEQ ID 005, SEQ ID 006, SEQ ID 009, SEQ ID 010, SEQ ID 012, and SEQ ID 013. Additionally, while the sequences are disclosed 5′-3′, the reversed sequences are contemplated where appropriate, and are considered to be disclosed herein. For example, an exemplary sequence disclosed as 5′-AGTATAAATAT-3′ is understood to additionally include the sequence 5′-TATAAATATGA-3′.

As has been described, thermal melt calibrators can be designed to have a variety of melt profiles at a variety Tms. In one aspect, for example, the calibrator can have a melt profile with a peak at a temperature of from about 60° C. to about 75° C. In another aspect, the calibrator can have a melt profile with a peak at a temperature of from about 64° C. to about 70° C. Such “low melt” calibrators can generally be A-T rich. As such, A-T rich sequences having a limited alternative base pairings sufficient to allow hybridization can thus be used to construct low melt calibrators, and are thus considered to be within the present scope. In some examples, there may only be a single base pairing position that allows hybridization of the stem sequences.

In one specific aspect, such a low melt calibrator can include a first nucleotide sequence selected from SEQ ID 001, SEQ ID 002, SEQ ID 009, SEQ ID 010, or the like. In another specific aspect, the low melt calibrator can include a first nucleotide sequence selected from SEQ ID 001 or SEQ ID 002. Furthermore, in one specific aspect, the loop sequence for the low melt calibrator can be SEQ ID 003 or similar. Furthermore, in one aspect the first nucleotide sequence can be SEQ ID 001, the second nucleotide sequence can be SEQ ID 002, and the loop sequence can be SEQ ID 003. In yet another aspect the first nucleotide sequence can be SEQ ID 009, the second nucleotide sequence can be SEQ ID 010, and the loop sequence can be SEQ ID 003.

Additionally, in another aspect the calibrator can have a melt profile with a peak at a temperature of from about 85° C. to about 105° C. In another aspect, the calibrator can have a melt profile with a peak at a temperature of from about 90° C. to about 98° C. Such “high melt” calibrators can generally be G-C-T rich. As such, G-C rich sequences having a limited alternative base pairings sufficient to allow hybridization can thus be used to construct high melt calibrators, and are thus considered to be within the present scope. In some examples, there may only be a single base pairing position that allows hybridization of the stem sequences.

In one specific aspect, such a high melt calibrator can include a first nucleotide sequence selected from SEQ ID 005, SEQ ID 006, SEQ ID 012, SEQ ID 013, or the like. In another specific aspect, the high melt calibrator can include a first nucleotide sequence selected from SEQ ID 005 or SEQ ID 006. Furthermore, in one specific aspect, the loop sequence for the high melt calibrator can be SEQ ID 007 or similar. Furthermore, in one aspect the first nucleotide sequence can be SEQ ID 005, the second nucleotide sequence can be SEQ ID 006, and the loop sequence can be SEQ ID 007. In yet another aspect the first nucleotide sequence can be SEQ ID 012, the second nucleotide sequence can be SEQ ID 013, and the loop sequence can be SEQ ID 007.

It is noted that the above thermal calibrators can be used singly, or in combination with other stem loop calibrators. For example, in some cases a single calibrator sequence, either low melt or high melt, can be utilized in an analysis reaction. In another aspect, both low melt and high melt calibrators can be utilized. Additionally, multiple calibrators of similar Tm ranges (e.g. low melt calibrators) having different sequences can be utilized together.

In a further aspect, the present disclosure provides assay compositions. In one aspect, for example, an assay composition can include a sample of genomic DNA, at least one pair of primers specific to a DNA sequence of interest, and at least one polynucleotide thermal melt calibrator. In one aspect, the polynucleotide thermal melt calibrator can be SEQ ID 004. In another aspect, the polynucleotide thermal melt calibrator can be SEQ ID 008. In yet another aspect, the polynucleotide thermal melt calibrator can be SEQ ID 011. In a further aspect, the polynucleotide thermal melt calibrator can be SEQ ID 014. Non-limiting genes of interest can include SMN, OTC, PAH, CHK2, and the like.

### EXAMPLES

The following examples are provided to promote a more clear understanding of certain embodiments of the present disclosure, and are in no way meant as a limitation thereon.

### Example 1

Hairpin calibrators were compared experimentally with Idaho Technology's (Salt Lake City, Utah) complementary oligonucleotide calibrators (Gundry et al., NAR (2008) 36(10): 3401-8). Six different temperature shift neutral SNPs were selected for comparison. Results obtained indicate melt temperature calibration using the present stem loop calibrators perform as well as complementary oligonucleotide calibrators. However, when complementary oligonucleotide calibrators were used at higher concentrations amplification of target genes was delayed. It is believed that this delay is due to the presence of 4 oligonucleotides (low and high calibrators) in addition to the gene specific primers and genomic DNA during PCR and HRM resulting in reactions of increased complexity and more susceptible to failure. The low cost for synthesis and concentration independence of the present calibrators facilitate robust signal detection across platforms and make them very useful in the clinical laboratory setting.

Methods and Materials

Genomic DNA was isolated from:

1) Whole blood by MagNA Pure: Five mL of peripheral blood were obtained by venipuncture from 16 healthy volunteers and genomic DNA (gDNA) was isolated using the MAGna Pure Compact system (Roche, Indianapolis, Ind.).

2) Formalin-Fixed Paraffin Embedded (FFPE) bone marrow biopsies: A total of 10 FFPE and decalcified bone marrow trephine biopsies submitted to our laboratory between 2000 and 2012 were obtained for comparison with fresh samples. DNA from FFPE bone marrow trephine biopsies was isolated after deparaffinization with mineral oil using the DNeasy Micro Kit (Qiagen, Valencia, Calif.).

3) Dried Blood Spot (DBS): Sixteen de-identified whole blood specimen from the ARUP Clinical Molecular Genetics Laboratory were spotted on Whatman 903 paper (50 μL) and set to dry at room temperature overnight. A 3.2 mm round punch is utilized in DNA extraction in two washes with DNA Purification Solution 1 (Qiagen, catalog no. 159992) and final elution in DNA Elution Solution 2 (Qiagen catalog no. 159994) as described (Heath et al., APLM (1999); 123: 1154-60).

Following extraction, gDNA quality (1.8≦O.D. 260/280 ratio≦2.0) and quantity was determined using ultraviolet (UV) spectroscopy (NanoDrop® ND-8000; Thermo Scientific, Wilmington, Del.). For use in PCR and HRM, gDNA aliquots were adjusted to a final concentration of 20 ng/μL.

All specimens were collected and tested under a University of Utah Institutional Review Board (IRB) approved research protocol (7275).

Oligonucleotides:

Oligonucleotides were obtained from Integrated DNA Technologies (IDTDNA, Coralville, Iowa).

1) Temperature Calibrators

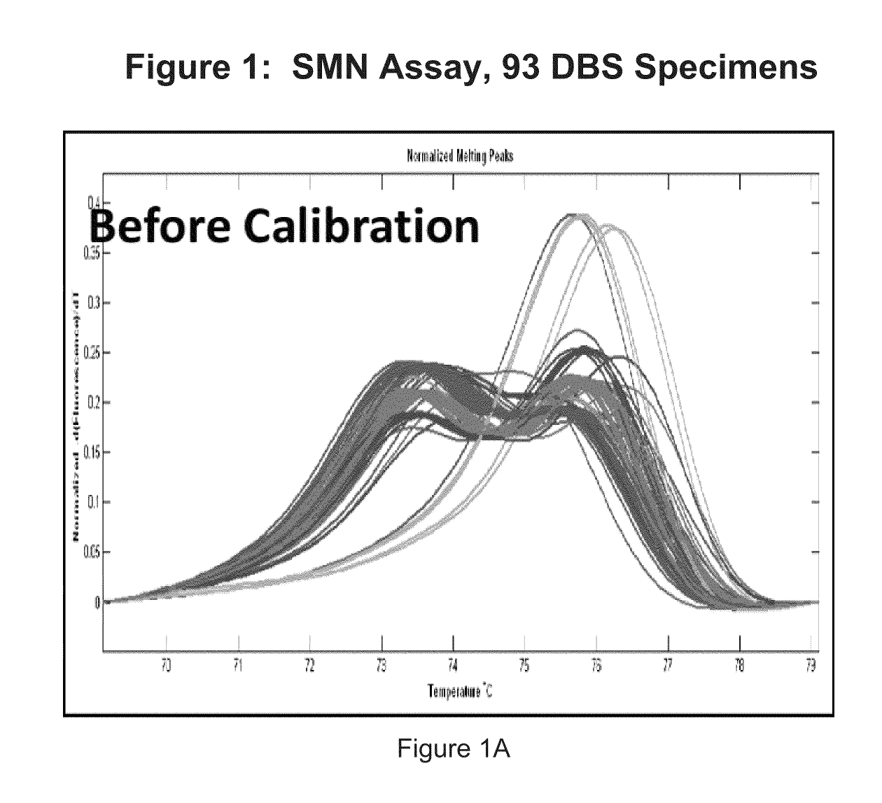

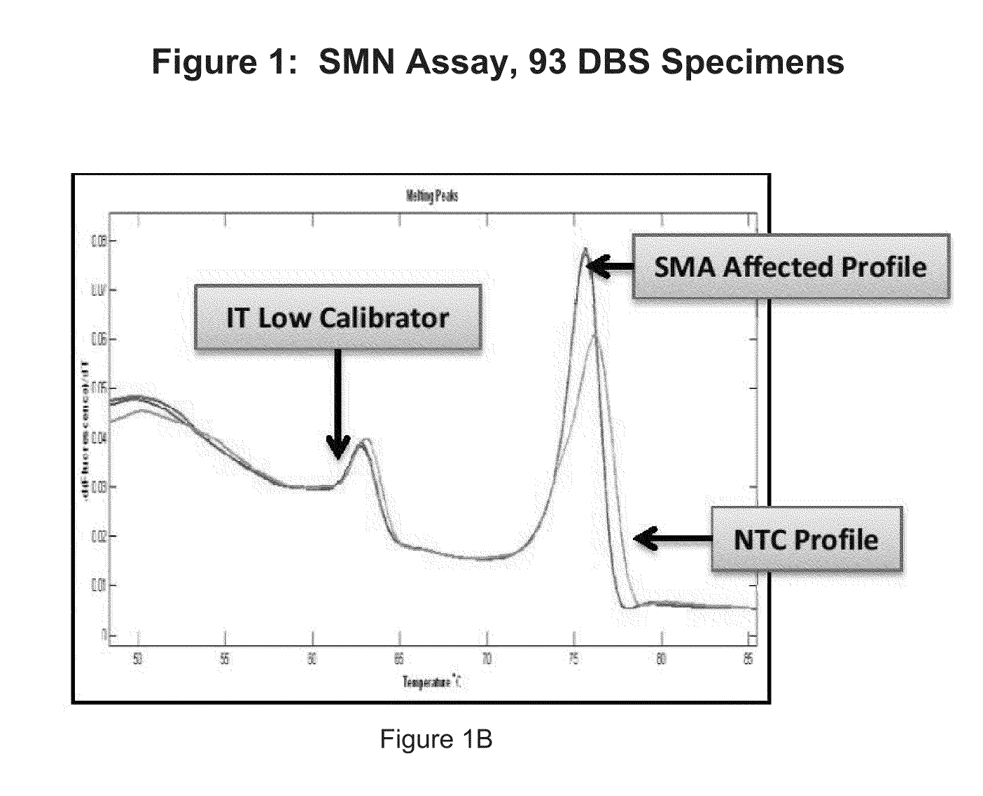

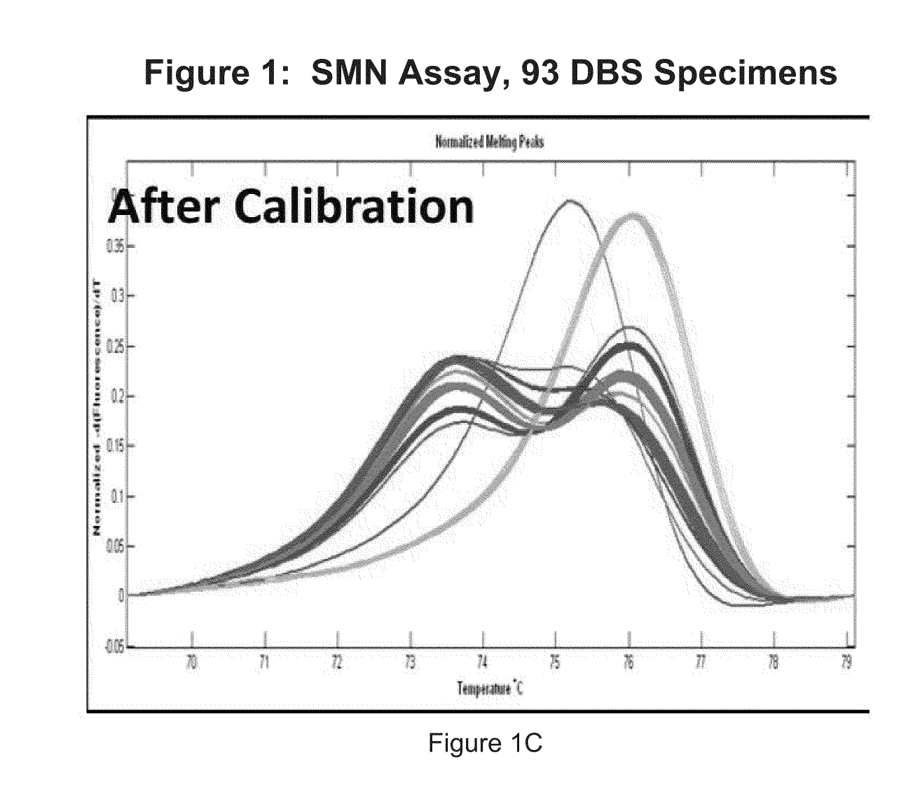

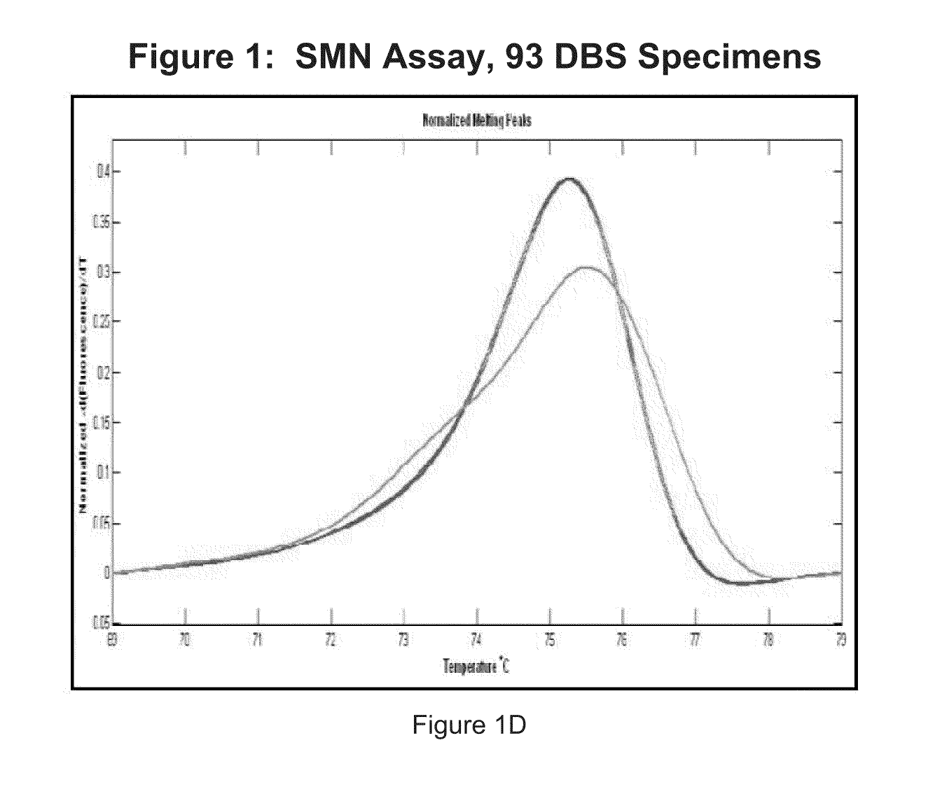

HRM melting temperature was adjusted by use of internal oligonucleotide, double stranded2; 3 or novel stem-loop, calibrators. Low and high Tm double stranded oligonucleotide calibrators were as described by Gundry and colleagues. Briefly, both sense and antisense double stranded oligonucleotide calibrators (Table 2) were 3′ end blocked by a propanediol spacer (3SpC3) to prevent extension by DNA polymerase and polyacrylamide gel electrophoresis (PAGE) purified after synthesis. Stem-loop oligonucleotide calibrators (FIG. 1) were designed using Oligo 7 (Molecular Biology Insights, Cascade, Colo.), OligoAnalyzer 3.1 (IDTDNA, Coralville, Iowa), and UnaFold (The RNA Institute, College of Arts and Sciences, University at Albany). Stem-loop calibrators were PAGE-purified following synthesis.

2) Gene Specific Oligonucleotides

Four different temperature-shift neutral SNPs were from Gundry et al., NAR (2008) 36(10):3401-8. They include four variants for the following genes: ornithine transcarbamylase (OTC c.299-8T>A), mutator homolog 2 (MSH2 rs12998837), phenylalanine hydroxylase (PAH rs772897), and checkpoint 2 (CHK2 rs9608698). The spinal muscular atrophy assay is based on genotyping nucleotide c.840 of the highly homologous, AT-rich, survival motor neuron genes, SMN1 (C) and SMN2 (T).

Oligonucleotide sequences are shown in Table 1.

TABLE 1
Gene Amplification Primers

| | | | Primer | Amplicon |
|---|---|---|---|---|
| | | | Length | Length |
| Gene | Variant | Primer Sequence (5′-3′) | (bp) | (bp) |
| SMN | c.840 C > T | FWD: TTCCTTTATTTTCCTTACAGGGTTT | 25 | 52 |
| | rs not assigned | (SEQ ID 15) | | |
| | | REV: ACCTTCCTTCTTTTTGATTTTGTCT | 25 | |
| | | (SEQ ID 16) | | |
| OTC | c.299-8 T > A | FWD: TCCACTTTAGTTGTTTTTTCAAAATGAT | 28 | 68 |
| | rs not assigned | (SEQ ID 17) | | |
| | | REV: CCCAGAAGTGCAAAGCCTAC | 20 | |
| | | (SEQ ID 18) | | |
| PAH | c.1155 C > G | FWD: AAATTACACTGTCACGGAGTTCCA | 24 | 59 |
| | rs 772897 | (SEQ ID 19) | | |
| | | REV: CATCATTAAAACTCTCTGCCACGTAATA | 28 | |
| | | (SEQ ID 20) | | |
| CHK2 | 1-3850 G > C | FWD: AGTGAAGTGACGCATGTAATACTC | 24 | 86 |
| | rs 9608698 | (SEQ ID 21) | | |
| | | REV: ACTTCTCTGCATGGCTTTCC | | |
| | | (SEQ ID 22) | | |

PCR Conditions:

PCR was performed in 10 μL containing 1× LCGreen Plus, 0.1 U/μL KlenTaq1, 0.2 mM dNTPs, and 2 mM MgCl2. Either double-stranded or stem-loop temperature calibrators were added to a final concentration of 0.0504 unless otherwise specified. Genomic DNA was added to a final concentration of 2 ng/μL. A 20 μL mineral oil overlay is included in all reactions to prevent evaporation.

PCR reactions were performed using a C-1000 thermal cycler (Bio-Rad, Hercules, Calif., USA).

PCR conditions are shown in Table 2.

TABLE 2
Complementary and Stem Loop oligonucleotide Calibrator Sequences

| | | | Size | T_{d} | |
|---|---|---|---|---|---|
| | Name | Sequence (5′-3′) | (bp) | Loc | 3′ Block |
| ds-Cal | Low Cal | SEQ ID 009- | 50 | 62 | 3SpC3 |
| | 1 | TTAAATTATAAAATATTTATAATATTAATTATATATATATAAATATAATA | | | |
| | Low Cal | SEQ ID 010- | 50 | 62 | 3SpC3 |
| | 2 | TATTATATTTATATATATAATTAATATTATAAATATTTTATAATTTAA | | | |
| | High Cal | SEQ ID 012- | 63 | 92 | 3SpC3 |
| | 1 | GCGCGGCCGGCACTGACCCGAGACTCTGAGCGGCTGCTGGAGGTGCGGAA | | | |
| | High Cal | SEQ ID 013- | 63 | 92 | 3SpC3 |
| | 2 | CCCGCCCCTCCGCTTCCGCACCTCCAGCAGCCGCTCAGAGTCTCGGGTC | | | |
| Cal.Lp | Cal. Lp. | SEQ ID 004- | 83 | 66 | Nono |
| | Low | TATAATAAATAAGTATAAATATAATAGAATAATATAATCCCCCCCATTAT | | | |
| | v4.0 | ATTATTCTATTATATTTATACTTA TTTATTATA | | | |
| | Cal. Lp. | SEQ ID 008- | 60 | 94 | None |
| | High | GGC CCG GTC GCG CAG AGG GTC CCA CGA CCT TCG TCG | | | |
| | V | TGG GAC CCT CTG CGC GAC CGG GCC | | | |

High-Resolution Melt:

Post-PCR high-resolution melt was performed on the LightScanner 96 (BioFire Diagnostics Inc., Salt Lake City, Utah, USA). Temperature dependent DNA melting profiles were collected between 55-98° C. Melting profiles were analyzed using the LightScanner 96 software. Temperature calibration was adjusted using the melting profiles obtained for either double-stranded or stem-loop calibrators as described in the LightScanner 96 software. Specimens were genotyped after temperature calibration and results compared to known controls.

Results:

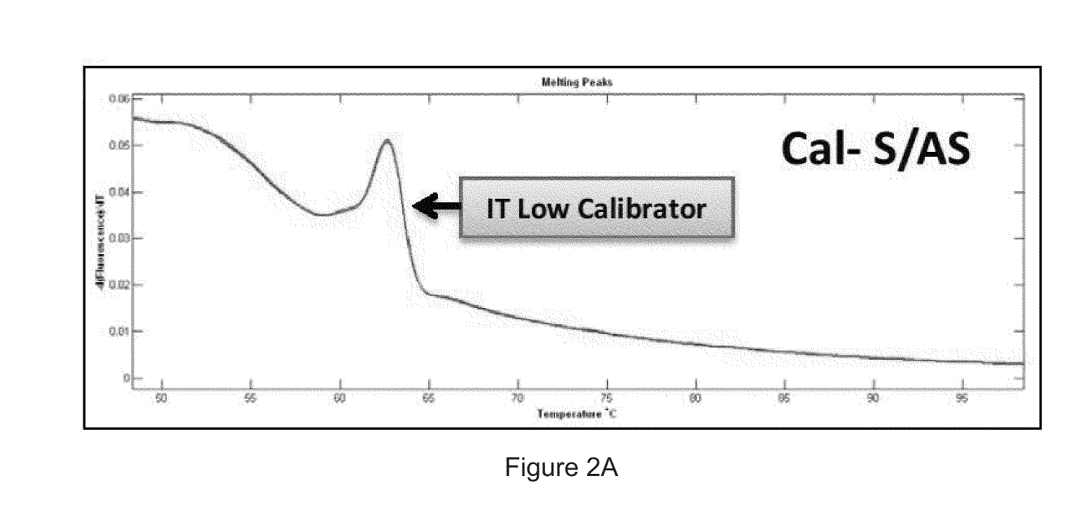

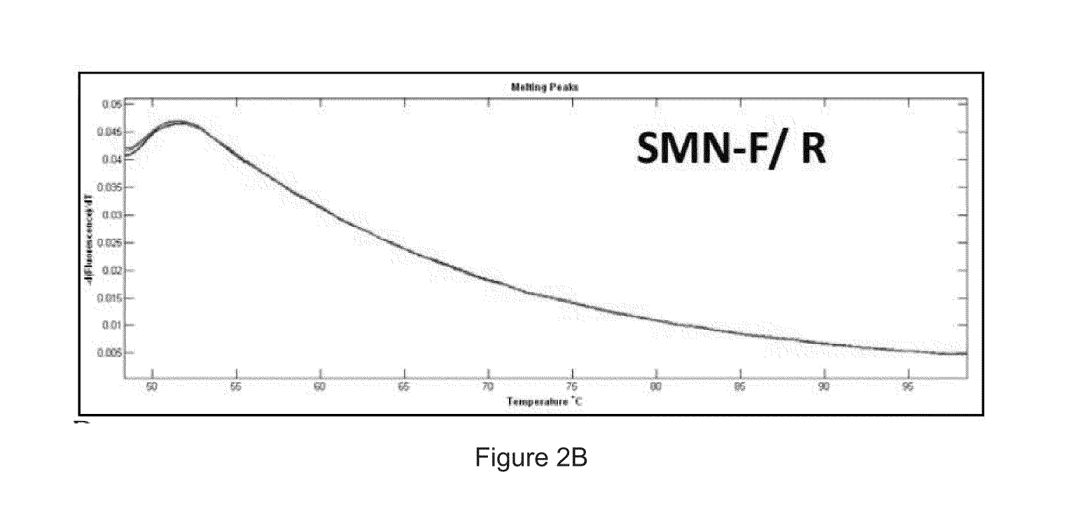

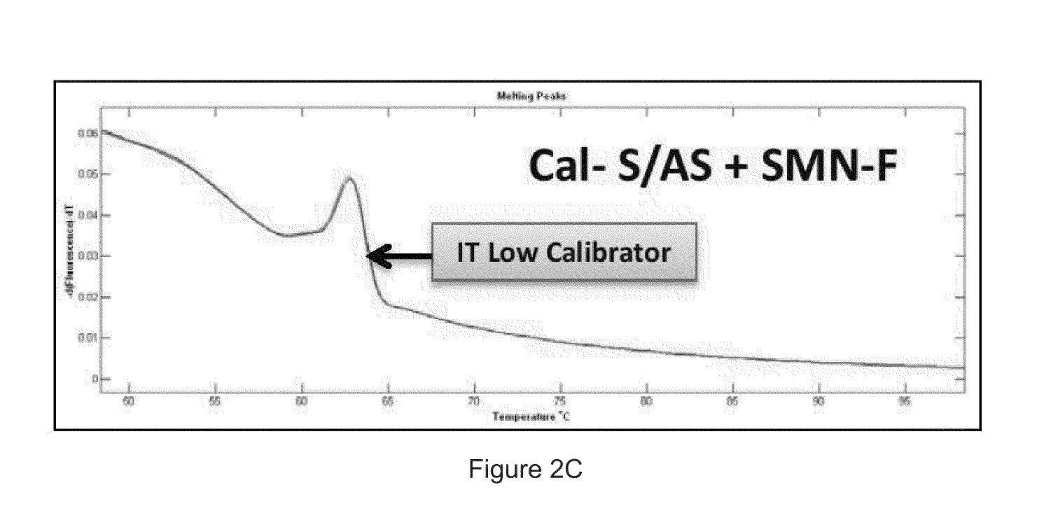

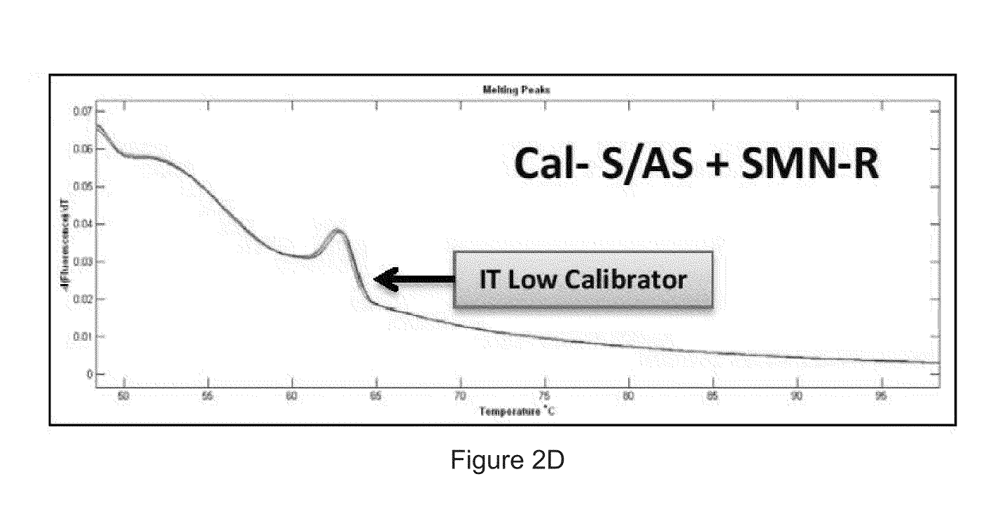

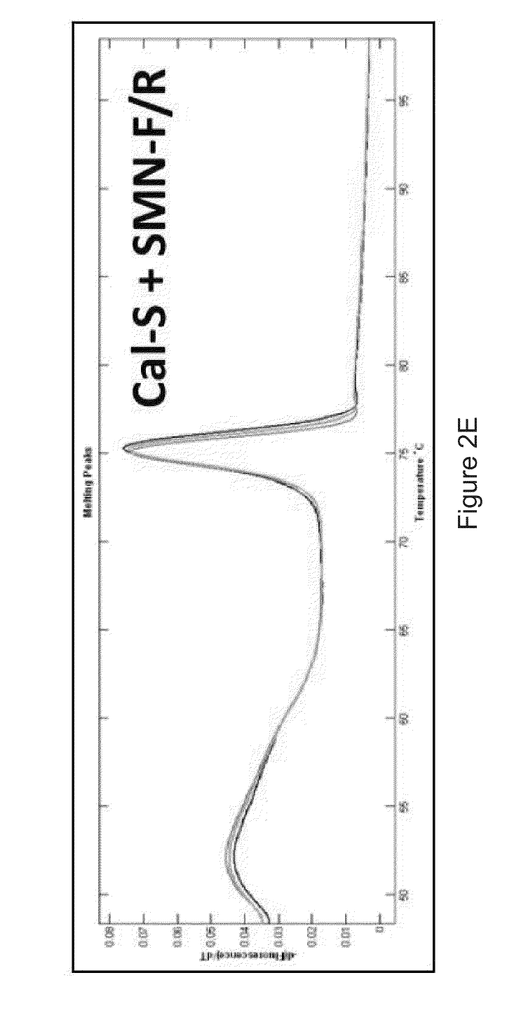

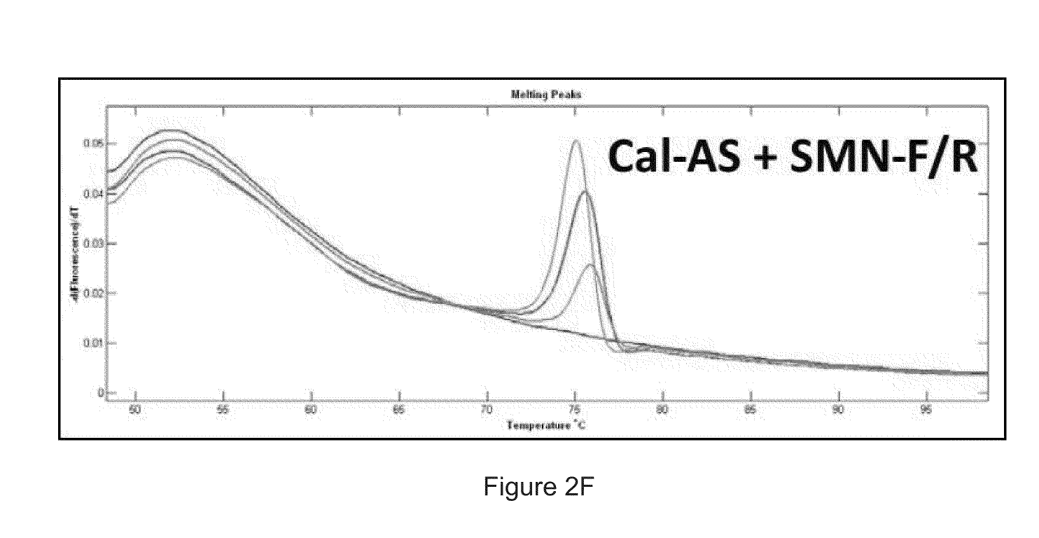

An HRM assay for spinal muscular atrophy (SMA) based on PCR amplification using primers designed to flank site c.840 of the highly homologous SMN1 and SMN2 genes was recently reported. The presence and absence of a C nucleotide at position c.840 produced two distinguishable melting profiles post-PCR. While amplifications were robust and post-melting profiles were easily calibrated using double-stranded calibrator, significant PCR amplification in the no template control (NTC) reactions was observed (FIG. 1). Moreover, amplicon size in the NTC control was in agreement with that expected for the SMN products and melt profiles were in the same temperature range as well (FIG. 1). Multiple reasons are proposed to explain this observation, the most logical is reagent or materials contamination and less likely primer dimer formation. In order to address these possibilities, new SMN primers, double stranded calibrators, materials and reagents were ordered. Lab benches and pipettes were thoroughly decontaminated. However, in spite of all these approaches, as well as, setting up reactions in different laboratories/buildings, continued PCR amplification and melt profiles were noted in the NTC controls (data not shown). Based on extensive troubleshooting, suspicion was raised that a non-specific amplification was occurring when double stranded calibrator and SMN primers were present in the same reaction. In order to investigate the possibility that the double stranded calibrator was the cause of NTC amplification several NTC reactions using all possible combinations of sense (S) and/or antisense (AS) calibrator with forward (SMN-F) and/or reverse (SMN-R) primers (FIG. 2 A-F) were set up. First priority was given to ascertaining that the materials and reagents were not contaminated. This was addressed by running reactions with calibrators alone (FIG. 2A) or SMN primers alone (FIG. 2B). No PCR amplification/melt profile was observed. Next, amplification of SMN-F (FIG. 2C) or SMN-R (FIG. 2D) primers in the presence of S and AS calibrator mix was tested. No PCR amplification/melt profile was observed. Finally, amplification of S (FIG. 2E) or AS (FIG. 2F) calibrator strands in the presence of SMN-F and SMN-R primer mix was tested. In contrast to the previous results, amplification for both S or AS calibrators in the presence of both SMN primers was detected.

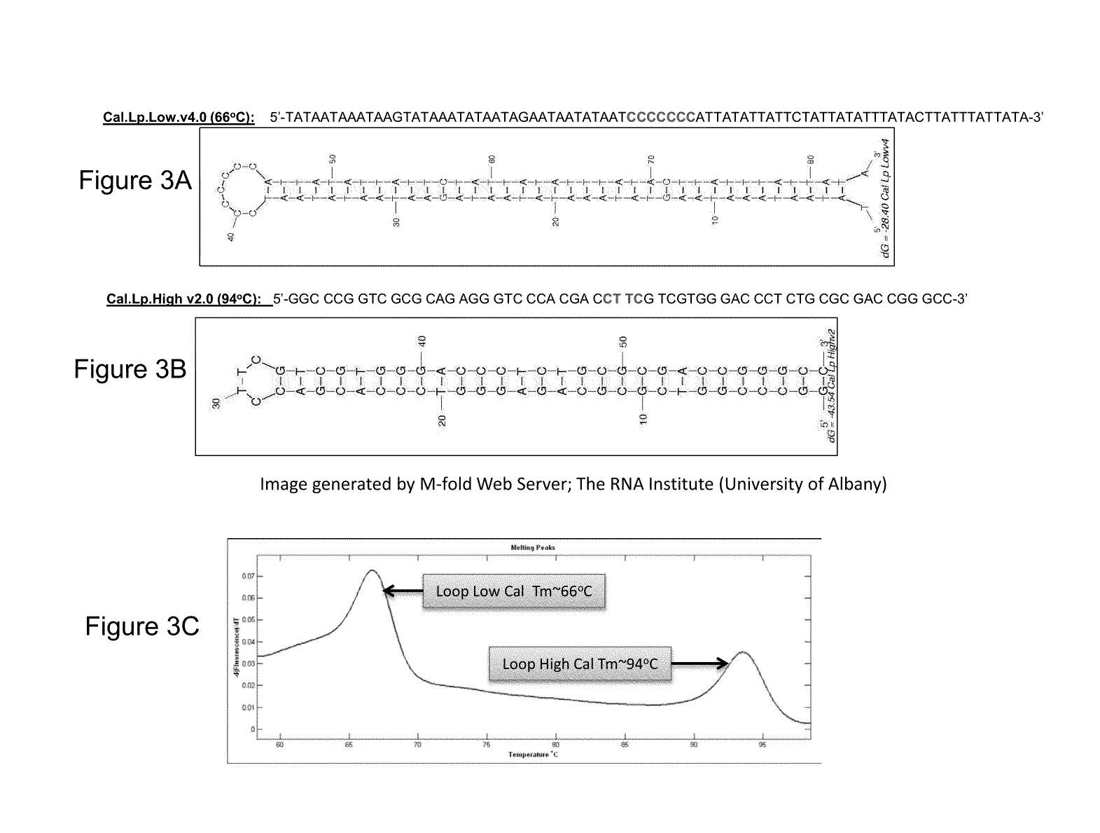

Based on the problems observed with the ds.Cal, it was decided to design novel calibrators based on a single oligonucleotide. Similar to the ds.Cal, these novel calibrators should melt at specific Tm's, must be able to flank most amplicons, have to be detectable by fluorescence intercalating dyes, and should not interfere with PCR. The most appropriate design fitting all requirements was a calibrator based on a stem loop structure. Two such calibrators were created, one with a low Tm=66° C. (Low.Cal.Lp) and another with a high Tm=94° C. (High.Cal.Lp) shown in FIG. 3A. Melting profiles for a mixture of both stem-loop calibrators is shown in FIG. 3B.

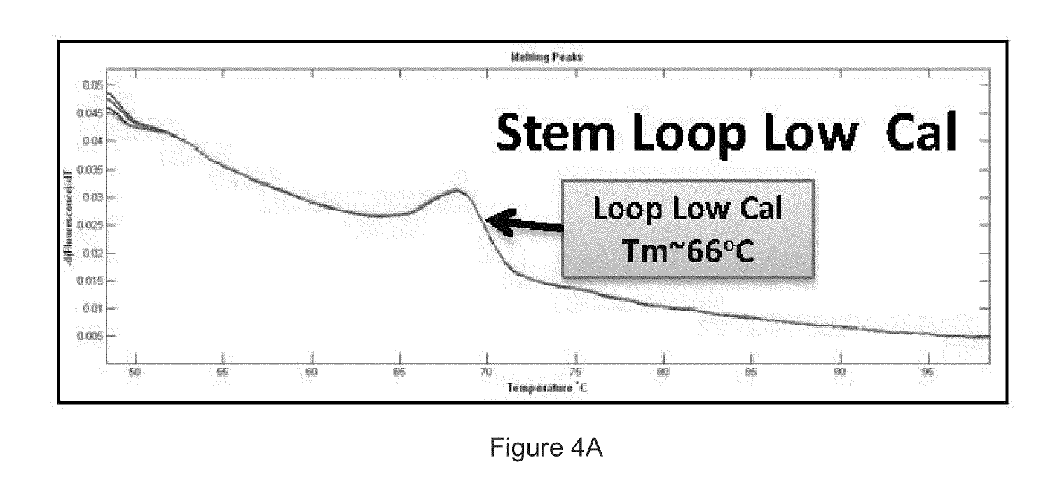

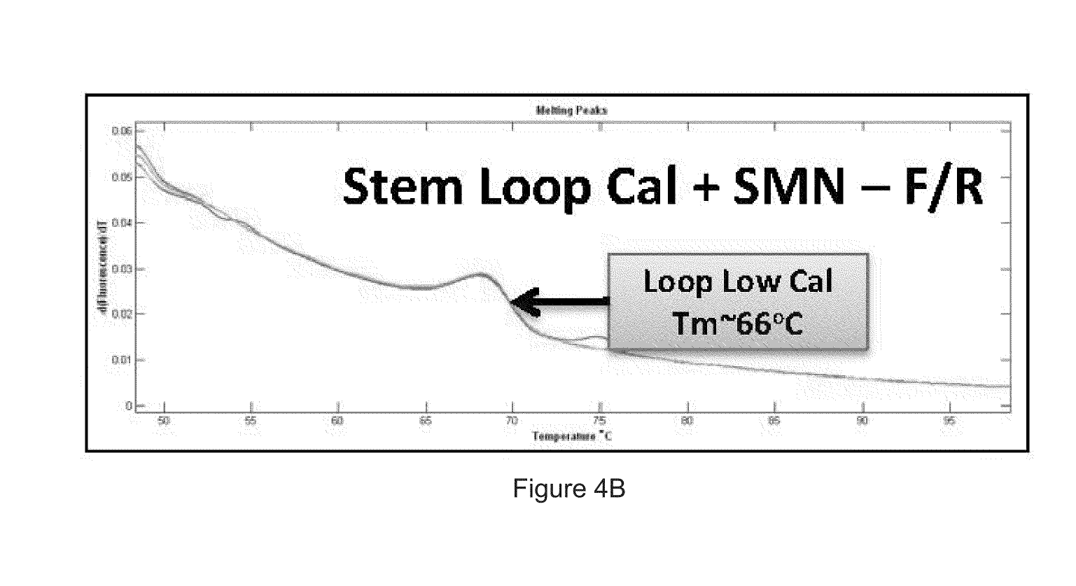

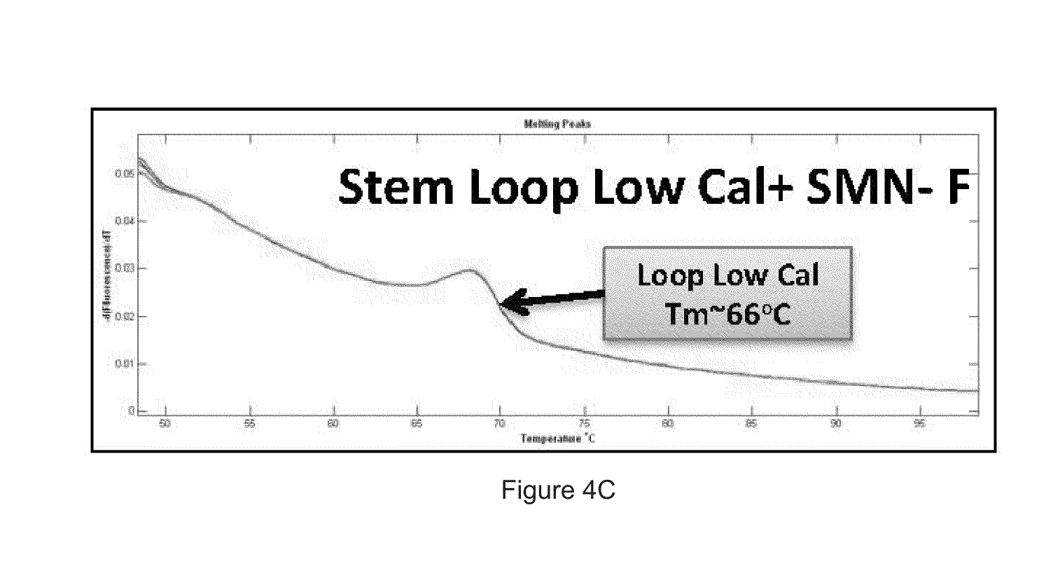

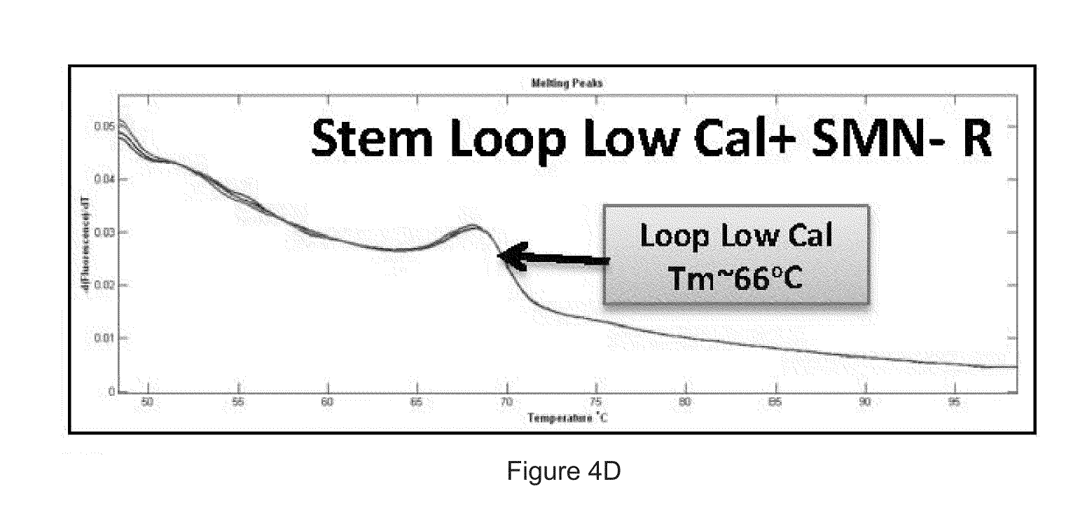

Since this new design was intended to solve the problems observed with the NTC amplification using the SMN primers, the next step was to test them under the same conditions as described above for the ds.Cal. Hence, NTC reactions were set up using all possible combinations of the Low.Cal.Lp calibrator with SMN-F and/or SMN-R gene specific primers (FIG. 4A-D). No PCR amplification/melt profile was observed at 75° C. in any of the reactions; only the melting peak for the Low.Cal.Lp is seen.

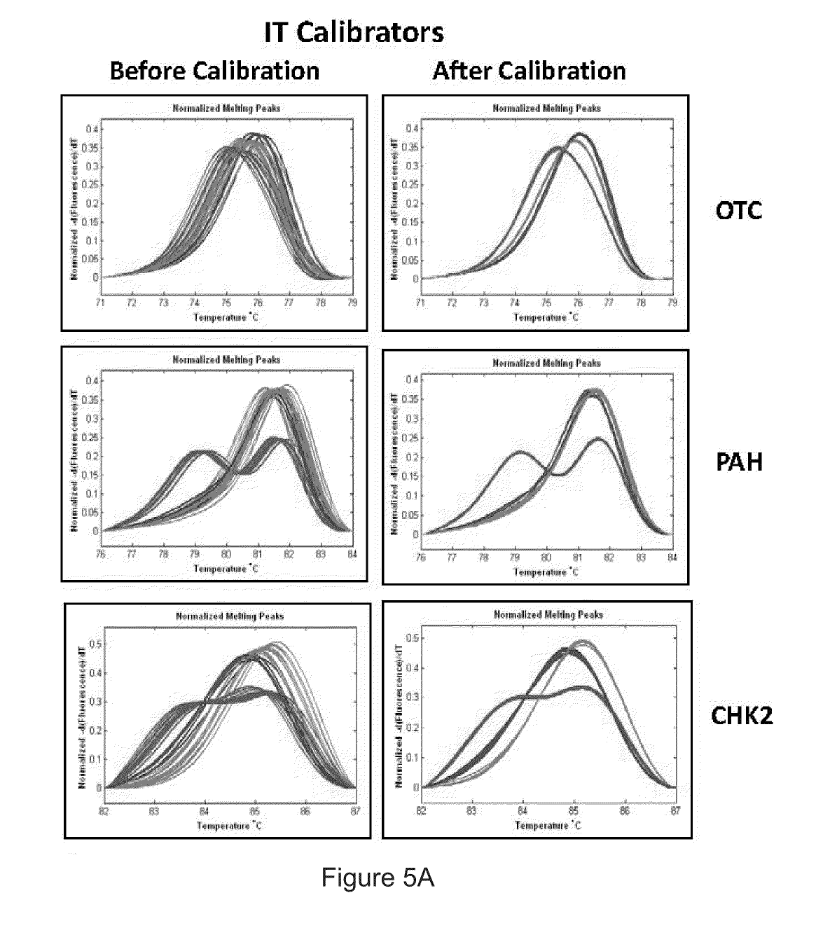

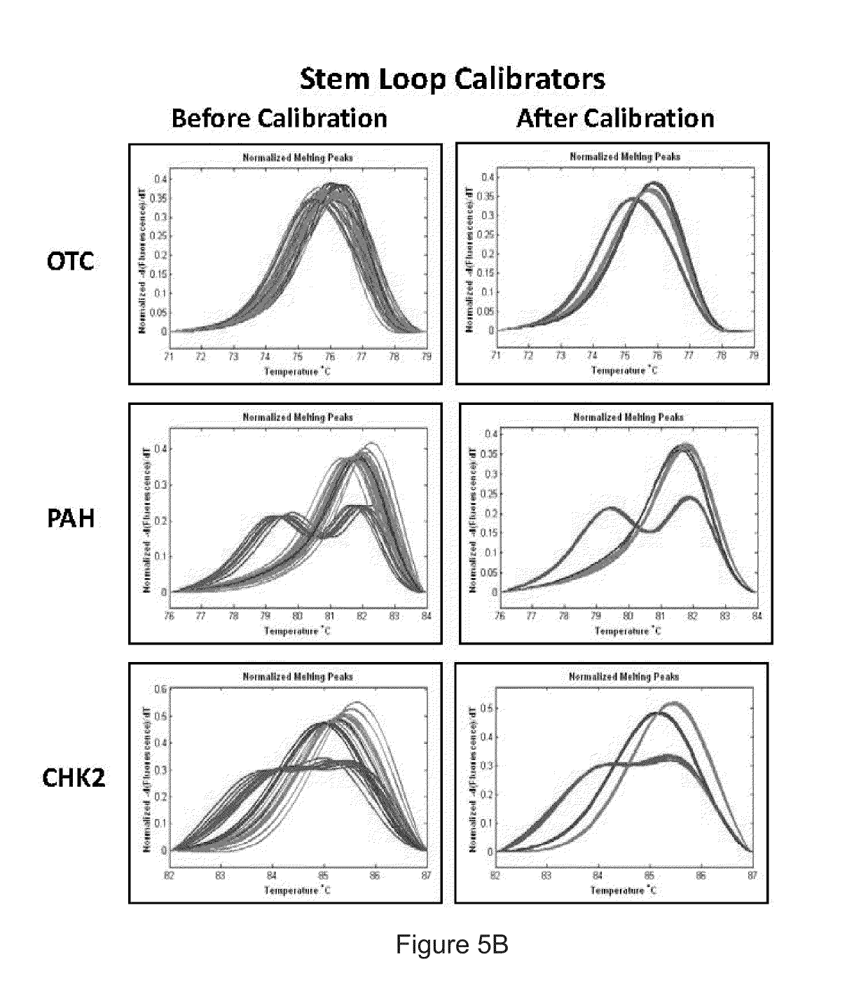

In order to further characterize these calibrator designs a comparison was made of PCR/HRM calibrated with either ds.Cal or Cal.Lp for genotyping of the SMN c.840 position (Dobrowoski), as well as, three temperature shift neutral polymorphisms in the genes for, CHK2, OTC, and PAH (Gundry). Derivative melting curves for the four polymorphisms, before and after calibration with either ds.Cal (Panel 1) or Cal.Lp (Panel 2) is shown in FIG. 5. Melting profiles demonstrate that clear discrimination between homozygote peaks is only achieved post-calibration, independent of which calibrator, ds.Cal or Cal.Lp, is used (FIG. 5). Moreover, we have also assessed the utility of our novel design for the purposes of calibration and normalization of DNA specimens extracted from different sources. Derivative melting curve profiles for the three temperature shift neutral polymorphisms in the genes for, CHK2, OTC, and PAH were done using DNA isolated from dried blood spots, fresh whole blood, and formalin fixed paraffin embedded specimens (FIG. 5). As already mentioned above, no difference in calibration efficiency between ds.Cal and Cal.Lp was observed.

Discussion:

Based on the foregoing, the following conclusions were reached.
In DNA, the favored structure is the stem loop versus the double strand with internal bulge.
As illustrated here, Gibbs free energy, or the delta G is more negative for the stem loop thus, more favorable.

The stem loop structure is more stable than the double stranded linear bulge structure because the stem loop can twist around itself and form a tighter structure.
This is the basic shape of a stem loop which can twist to form this structure and can twist around itself to form a super coil.

1) Target genes OCT, MSH2, PAH, and CHK2 were evaluated and found both IT and stem loop to do well in calibration.

2) Amplification for AT-rich targets, such as the SMN gene, cross reaction with IT low calibrator during PCR and generate NTC melts whereas the stem loop low calibrator do not display the profile.

3) The IT high melting curve derivative is suggestive of heteroduplex sequence and the stem loop melting curve has an appearance of a homoduplex.

4) IT calibrators cost 2.5 times more than the stem loop calibrators.

5) No concerns were formed regarding titration of strand concentrations with Cal.Lp compared to ds.Cal.

6) While ds.Cal is dependent on concentration and diffusion of ss.oligos for base-pairing, the Cal.Lp always has complementary strands together making it independent of concentration or diffusion.

7) However, Cal.Lp do not require a 3′-end block, PAGE purification is still recommended based on the size of oligos and due to possibility of incomplete synthesis.

### Example 2

Table 3 shows gene specific PCR protocols for various genes of interest.

TABLE 3
Gene Specific PCR Protocol

| | Primer | Initial | | Denaturation | | Annealing | | |
|---|---|---|---|---|---|---|---|---|
| | Conc. | Temp | Time | Temp | Time | Temp | Time | |
| Gene | (μM) | (° C.) | (mins) | (° C.) | (mins) | (° C.) | (mins) | Cycle (s) |
| SMN | 0.10 | 95 | 1:00 | 95 | 0:20 | 60 | 0:30 | 45 |
| OTC | 0.20 | 95 | 2:00 | 94 | 0:30 | 65 | 0:30 | 50 |
| PAH | 0.20 | 95 | 2:00 | 94 | 0:30 | 67 | 0:30 | 45 |
| CHK2 | 0.25 | 95 | 2:00 | 94 | 0:30 | 66 | 0:30 | 45 |

It is understood that the above-described methods, arrangements and/or modes of operation are only illustrative of preferred embodiments of the present invention. Numerous modifications and alternative arrangements may be devised by those skilled in the art without departing from the spirit and scope of the present invention and the appended claims are intended to cover such modifications and arrangements. Thus, while specific embodiments of the present invention have been described with particularity and detail, it will be apparent to those of ordinary skill in the art that variations including, but not limited to, variations in size, amount, materials, function and manner of operation and use may be made without departing from the principles and concepts set forth herein.

What is claimed is:

1. A polynucleotide thermal melt calibrator having a stem-loop configuration, comprising:
a first stem region having a first nucleotide sequence;
a second stem region having a second nucleotide sequence, wherein the second nucleotide sequence is sufficiently complimentary to the first nucleotide sequence to at least substantially hybridize and at least substantially denature across a temperature range of about 10° C. or less;
a loop region joining the first stem region and the second stem region, the loop region having a loop sequence and a loop length to allow hybridization of the first stem region and the second stem region;
a hybridization indicator associated with at least one of the first stem region, the second stem region, or the loop region.

2. The calibrator of claim 1, wherein the second nucleotide sequence is sufficiently complimentary to the first nucleotide sequence to substantially hybridize and substantially denature across a temperature range of about 5° C. or less.

3. The calibrator of claim 1, wherein the first nucleotide sequence is selected from the group consisting of SEQ ID 001, SEQ ID 002, SEQ ID 005, SEQ ID 006, SEQ ID 009, SEQ ID 010, SEQ ID 012, and SEQ ID 013.

4. The calibrator of claim 1, wherein the calibrator has a melt profile with a peak at a temperature of from about 60° C. to about 75° C.

5. The calibrator of claim 4, wherein the calibrator has a melt profile with a peak at a temperature of from about 64° C. to about 70° C.

6. The calibrator of claim 4, wherein the first nucleotide sequence is selected from the group consisting of SEQ ID 001, SEQ ID 002, SEQ ID 009, and SEQ ID 010.

7. The calibrator of claim 6, wherein the loop sequence is SEQ ID 003.

8. The calibrator of claim 4, wherein the first nucleotide sequence is SEQ ID 001, the second nucleotide sequence is SEQ ID 002, and the loop sequence is SEQ ID 003.

9. The calibrator of claim 1, wherein the calibrator has a melt profile with a peak at a temperature of from about 85° C. to about 105° C.

10. The calibrator of claim 9, wherein the calibrator has a melt profile with a peak at a temperature of from about 90° C. to about 98° C.

11. The calibrator of claim 9, wherein the first nucleotide sequence is selected from the group consisting of SEQ ID 005 and SEQ ID 006.

12. The calibrator of claim 11, wherein the loop sequence is SEQ ID 007.

13. The calibrator of claim 9, wherein the first nucleotide sequence is SEQ ID 005, the second nucleotide sequence is SEQ ID 006, and the loop sequence is SEQ ID 007.

14. An assay composition, comprising:
a sample of genomic DNA;
at least one pair of primers specific to a DNA sequence of interest; and
a polynucleotide thermal melt calibrator as in claim 1.

15. The composition of claim 14, wherein the polynucleotide thermal melt calibrator is SEQ ID 004.

16. The composition of claim 14, wherein the polynucleotide thermal melt calibrator is SEQ ID 008.

17. The composition of claim 14, wherein the polynucleotide thermal melt calibrator is SEQ ID 011.

18. The composition of claim 14, wherein the polynucleotide thermal melt calibrator is SEQ ID 014.


SEQUENCE LISTING (22 sequences)

SEQ ID NO: 1 - DNA, 38 bp, Artificial Sequence
  Synthetic
tataataaataagtataaatataatagaataatataat

SEQ ID NO: 2 - DNA, 38 bp, Artificial Sequence
  Synthetic
attatattattctattatatttatacttatttattata

SEQ ID NO: 3 - DNA, 7 bp, Artificial Sequence
  Synthetic
ccccccc

SEQ ID NO: 4 - DNA, 83 bp, Artificial Sequence
  Synthetic
tataataaataagtataaatataatagaataatataatcccccccattatattattctattatatttatacttatttattata

SEQ ID NO: 5 - DNA, 28 bp, Artificial Sequence
  Synthetic
ggcccggtcgcgcagagggtcccacgac

SEQ ID NO: 6 - DNA, 28 bp, Artificial Sequence
  Synthetic
gtcgtgggaccctctgcgcgaccgggcc

SEQ ID NO: 7 - DNA, 4 bp, Artificial Sequence
  Synthetic
cttc

SEQ ID NO: 8 - DNA, 59 bp, Artificial Sequence
  Synthetic
gcccggtcgcgcagagggtcccacgaccttcgtcgtgggaccctctgcgcgaccgggcc

SEQ ID NO: 9 - DNA, 50 bp, Artificial Sequence
  Synthetic
ttaaattataaaatatttataatattaattatatatatataaatataata

SEQ ID NO: 10 - DNA, 50 bp, Artificial Sequence
  Synthetic
tattatatttatatatatataattaatattataaatattttataatttaa

SEQ ID NO: 11 - DNA, 107 bp, Artificial Sequence
  Synthetic
ttaaattataaaatatttataatattaattatatatatataaatataataccccccctattatatttatatatatataattaatattataaatattttataatttaa

SEQ ID NO: 12 - DNA, 63 bp, Artificial Sequence
  Synthetic
gcgcggccggcactgacccgagactctgagcggctgctggaggtgcggaagcggaggggcggg

SEQ ID NO: 13 - DNA, 63 bp, Artificial Sequence
  Synthetic
cccgcccctccgcttccgcacctccagcagccgctcagagtctcgggtcagtgccggccgcgc

SEQ ID NO: 14 - DNA, 130 bp, Artificial Sequence
  Synthetic
gcgcggccggcactgacccgagactctgagcggctgctggaggtgcggaagcggaggggcgggcttccccgcccctccgcttccgcacctccagcagccgctcagagtctcgggtcagtgccggccgcgc

SEQ ID NO: 15 - DNA, 25 bp, Artificial Sequence
  Synthetic
ttcctttattttccttacagggttt

SEQ ID NO: 16 - DNA, 25 bp, Artificial Sequence
  Synthetic
accttccttctttttgattttgtct

SEQ ID NO: 17 - DNA, 28 bp, Artificial Sequence
  Synthetic
tccactttagttgttttttcaaaatgat

SEQ ID NO: 18 - DNA, 20 bp, Artificial Sequence
  Synthetic
cccagaagtgcaaagcctac

SEQ ID NO: 19 - DNA, 24 bp, Artificial Sequence
  Synthetic
aaattacactgtcacggagttcca

SEQ ID NO: 20 - DNA, 28 bp, Artificial Sequence
  Synthetic
catcattaaaactctctgccacgtaata

SEQ ID NO: 21 - DNA, 24 bp, Artificial Sequence
  Synthetic
agtgaagtgacgcatgtaatactc

SEQ ID NO: 22 - DNA, 20 bp, Artificial Sequence
  Synthetic
acttctctgcatggctttcc